(19) United States
(12) Patent Application Publication
(10) Pub. No.: US 2016/0046554 A1
(43) Pub. Date: Feb. 18, 2016

(54) PROCESS FOR PRODUCING STYRENE

(71) Applicant: SHELL OIL COMPANY, Houston, TX (US)

(72) Inventor: Jean Paul Andre Marie Joseph Gishlain LANGE, Amsterdam (NL)

(73) Assignee: SHELL OIL COMPANY, Houston, TX (US)

(21) Appl. No.: 14/783,120

(22) PCT Filed: Apr. 9, 2014
(86) PCT No.: PCT/EP2014/057205

§ 371 Date: Oct. 8, 2015

(30) Foreign Application Priority Data
Apr. 12, 2013 (EP) 13163486.7

(51) Int. Cl. C07C 45/45; C07C 29/145; C07C 45/00; C07C 1/24

(52) U.S. Cl. CPC C07C 45/455; C07C 1/24; C07C 29/145; C07C 45/00

(57) ABSTRACT

The invention relates to a process for producing styrene, comprising reacting benzene and acetic acid into methyl phenyl ketone and converting the methyl phenyl ketone into styrene. Preferably, the methyl phenyl ketone is converted into styrene by converting the methyl phenyl ketone into methyl phenyl carbinol and converting the methyl phenyl carbinol into styrene.

### FIELD OF THE INVENTION

The present invention relates to a process for producing styrene.

### BACKGROUND OF THE INVENTION

It is known to produce styrene by alkylation of benzene with ethylene resulting in ethylbenzene, followed by dehydrogenation of ethylbenzene into styrene, as follows:

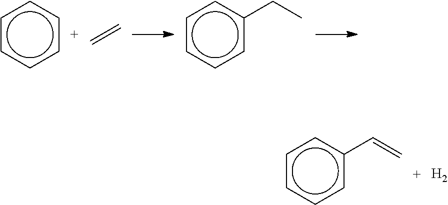

The required ethylene feedstock is also produced by a dehydrogenation reaction, that is to say by dehydrogenation of ethane. Therefore, on two occasions in the above styrene production process, highly oxidized components are produced by expensive dehydrogenation steps, namely in the dehydrogenation of ethane to ethylene and in the dehydrogenation of ethylbenzene to styrene. These dehydrogenation steps require a high capital expenditure.

A known alternative for the above-described direct styrene production process is a process wherein the above-mentioned second dehydrogenation step wherein ethylbenzene is directly converted to styrene is replaced by a sequence of three steps including the co-production of propylene oxide. These three steps comprise: (i) reacting ethylbenzene with oxygen or air to form ethylbenzene hydroperoxide, (ii) reacting the ethylbenzene hydroperoxide with propylene in the presence of an epoxidation catalyst to yield propylene oxide and methyl phenyl carbinol, and (iii) converting the methyl phenyl carbinol into styrene by dehydration using a dehydration catalyst. This alternative styrene production process is also commonly referred to as the SM/PO process for producing styrene monomer (SM) and propylene oxide (PO). As compared to the above-described direct styrene production process, by adding two additional steps in the SM/PO process an increase in capital expenditure is disadvantageously effected.

Thus, in said SM/PO process, styrene is produced via ethylbenzene hydroperoxide which is used to convert propylene into propylene oxide thereby also forming methyl phenyl carbinol, as follows:

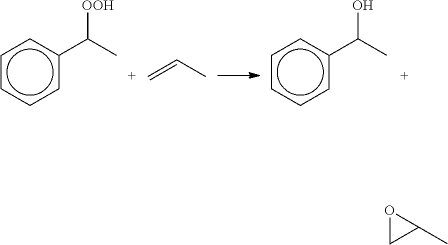

In a next step, said methyl phenyl carbinol is converted into styrene by dehydration, as follows:

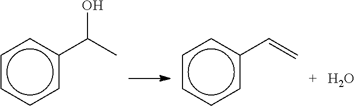

A further disadvantage of the production of styrene via the above-mentioned SM/PO process is that said ethylbenzene hydroperoxide is produced from ethylbenzene. As discussed above in connection with the direct production of styrene from ethylbenzene, production of ethylbenzene requires dehydrogenation of ethane to ethylene followed by alkylation of benzene with ethylene resulting in ethylbenzene. Therefore, in this case, a highly oxidized component is produced by an expensive dehydrogenation step, namely in the dehydrogenation of ethane to ethylene. This dehydrogenation step requires a high capital expenditure.

A still further disadvantage of the production of styrene via the above-mentioned SM/PO process is that styrene and propylene oxide are produced in a fixed SM:PO ratio of quantities. That is to say, in a case where the demand for styrene would increase as compared to the demand for propylene oxide, it may be that this increase in demand for styrene cannot be met in a case where total production capacity is maintained, or it may be that this increase in demand for styrene can be met in a case where total production capacity is increased but only at the cost of a remaining quantity of propylene oxide for which there is no demand. Both said situations are undesirable from both a technical and a commercial perspective.

Therefore, it is desired to provide a process for producing styrene which does not comprise any dehydrogenation step such as the above-discussed dehydrogenation steps from the known styrene production processes. Additionally, it is desired to provide a process for producing styrene which may compensate for any higher demand for styrene in the above-mentioned SM/PO process.

### SUMMARY OF THE INVENTION

Surprisingly it was found that the above-mentioned problems are solved by a process wherein benzene and acetic acid are reacted into methyl phenyl ketone, and the methyl phenyl ketone is converted into styrene. Advantageously, the present invention does not contain any dehydrogenation step for producing a highly oxidized component. One of the starting materials for the present invention is in fact acetic acid which in itself is already a highly oxidized component. Additionally, the present invention adds more flexibility for the above-mentioned SM/PO process in relation to any increase in the demand for styrene.

Accordingly, the present invention relates to a process for producing styrene, comprising reacting benzene and acetic acid into methyl phenyl ketone and converting the methyl phenyl ketone into styrene.

Apart from solving the above-mentioned problems relating to the known styrene production processes, the present invention also provides a process wherein acetic acid obtained as a side-product in biomass conversion processes can advantageously be used as a valuable feedstock to produce styrene. Thus, in the present process there is no need for first producing a highly oxidized component, such as acetic acid, by means of an expensive dehydrogenation step since in the present invention the acetic acid may simply originate from a biomass conversion process.

### BRIEF DESCRIPTION OF THE DRAWING

FIG. 1 is a diagram showing an embodiment of the present styrene production process wherein there is an integration with the above-mentioned SM/PO process.

### DETAILED DESCRIPTION OF THE INVENTION

In a first step of the present process for producing styrene, benzene and acetic acid are reacted into methyl phenyl ketone.

In the present invention, the acetic acid which is needed in the first step may originate from a biomass conversion process, that is to say a process wherein a renewable material (the biomass) is converted. In such biomass conversion process, the biomass is converted to mainly provide a fuel (e.g. ethanol) or other valuable chemicals. However, in such biomass conversion process, acetic acid may also be recovered or generated as a side-product for which an advantageous use is also desired. Thus, apart from solving the above-mentioned problems relating to the known styrene production processes, the present invention advantageously also provides a process wherein acetic acid obtained as a side-product in biomass conversion processes can be used as a valuable feedstock to produce styrene.

Specifically, in the present invention, the acetic acid may originate from biomass, preferably from a cellulosic material, such as a lignocellulosic material. Acetic acid is a minor, though significant component of lignocellulosic materials: it accounts for 2-3 wt. % of grasses and 3-6 wt. % of wood.

Various methods exist for obtaining acetic acid from biomass, preferably a cellulosic material, such as a lignocellulosic material.

A first method is to recover acetic acid by a treatment of the biomass at a relatively low temperature, preferably in the range of from 50 to 250° C., more preferably 100 to 200° C., in either liquid phase (also referred to as “pre-treatment”) or gas phase (also referred to as “torrefaction”) followed by extraction of the desired acetic acid. The remaining biomass can be used for other purposes, such as paper pulp, heat/power, biofuel or chemical manufacture. Such treatment is preferably carried out in an inert atmosphere and, optionally, in the presence of an acid or base. Torrefaction of willow wood releases about 3 wt. % of acetic acid.

A second method is to recover acetic acid by a treatment of the biomass at a relatively high temperature, preferably in the range of from 250 to 600° C., in either liquid phase (also referred to as “hydrolysis” or “liquefaction”) or gas phase (also referred to as “pyrolysis”) followed by extraction of the desired acetic acid. The remaining biomass can be used for other purposes, such as heat/power, biofuel or chemical manufacture. Such treatment is preferably carried out in an inert atmosphere and, optionally, in the presence of an acid or base. Hydrolysis of birch wood releases about 6 wt. % of acetic acid.

A third method is to convert sugars contained in biomass into acetic acid by fermentation.

Thus, on a large scale, for example in a plant wherein ethanol is produced as main product by converting cellulosic materials, such plant having a production capacity of 600 kt/a (kiloton per annum), it is expected that about 30 kt/a (5 wt. %) of acetic acid is produced.

At least for two reasons, torrefaction is a particularly interesting method for obtaining acetic acid from biomass, preferably a cellulosic material, such as a lignocellulosic material, and for providing the acetic acid thus obtained as a feedstock in the present process for producing styrene.

Firstly, by means of torrefaction acetic acid can be delivered in a stream having a reasonably high concentration of acetic acid, that is to say about 20 wt. % of acetic acid, upon recycling the gas effluent to the reactor to build up product, as for example disclosed in a publication on the world wide web which is entitled “Torrefaction for entrained-flow gasification of biomass” (report ECN-C-05-067), by P. C. A. Bergman et al., Energy research Centre of the Netherlands (ECN), ECN Biomass, web:

http://www.ecn.nl/docs/library/report/2005/c05067.pdf). On the other hand, in a wet lignocellulose conversion process wherein the lignocellulose is converted in a liquid (water), a stream containing only about 1 wt. % of acetic acid may be obtained. Such higher acetic acid concentration by torrefaction greatly facilitates recovery of the acetic acid and subsequent valorization thereof in the present process for producing styrene.

Secondly, torrefaction may become a major process since it is seen as serious candidate for making cellulosic materials a commodity for power generation plants or for BtL plants. “BtL” refers to the “Bio-to-Liquids” process that proceeds via gasification and Fischer-Tropsch synthesis. Replacing only 5% of coal consumption, which coal is globally used in an amount of about 5 Gt/a (gigaton per annum) to generate power, by cellulosic material consumption could already co-produce about 12.5 Mt/a (million ton per annum) of acetic acid (at 5 wt. % content in lignocellulose). On the other hand, the current global demand for acetic acid is about 11 Mt/a, which is less than the above-expected production of about 12.5 Mt/a of acetic acid. Currently, the major outlets for acetic acid are in producing vinyl acetate (33%), terephthalic acid (TPA) solvent (22%), acetate ester solvents (15%) and acetic anhydride (14%). However, by commercial application of the present process for producing styrene, a further advantageous outlet for acetic acid would be created by which a good portion of the above-expected production of about 12.5 Mt/a of acetic acid can effectively be absorbed.

In the present invention, the benzene which is needed in the first step may also originate from a biomass, more in particular from a biomass conversion process, just like the acetic acid as discussed above. In such way, advantageously in the present invention, fully bio-based styrene is produced. For example, benzene may be obtained from aqueous phase reforming of sugar, as for example disclosed in WO2011143391 and US2012019870. Further, benzene may be obtained from catalytic pyrolysis of lignocellulose, as for example disclosed by T. R. Carlson et al. in “Green Gasoline by Catalytic Fast Pyrolysis of Solid Biomass Derived Compounds”, ChemSusChem, 2008. Still further, benzene may be obtained from hydrodeoxygenation of pyrolysis oil or lignin, as for example disclosed by B. Valle in “Selective Production of Aromatics by Crude Bio-oil Valorization with a Nickel-Modified HZSM-5 Zeolite Catalyst”, Energy Fuels, 2010, 24, pages 2060-2070. All these routes give a stream of mixed aromatics, including benzene, which can further be treated to make further benzene, for example via hydrodealkylation or transalkylation.

In the present process for producing styrene, first benzene and acetic acid are reacted into methyl phenyl ketone. In this acylation reaction, water is co-produced as follows:

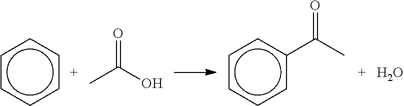

The above benzene acylation step, wherein benzene and acetic acid are reacted into methyl phenyl ketone, is known as such. For example, this reaction is disclosed by A. P. Singh et al. in J. Molec. Catal. A: Chem., 1997, 123, pages 141-147. Suitable conditions for effecting said reaction are disclosed in said publication, the entire disclosure of which is herein incorporated by reference.

In particular, in said publication in J. Molec. Catal. A: Chem., 1997, 123, pages 141-147, a 90% selectivity (based on acetic acid) is reported for a case where in the reaction of benzene with acetic acid a HZSM-5 zeolite was used as a catalyst and further the following conditions were applied: gas phase, 250° C., 1 atmosphere, benzene:acetic acid molar ratio of 1:2, and LHSV=1/h. Reported by-products are di- and tri-acylated products.

Generally, the above benzene acylation step, wherein benzene and acetic acid are reacted into methyl phenyl ketone, can be carried at a temperature in the range of from 150 to 350° C., preferably 200 to 300° C., and at a pressure ranging from atmospheric pressure to lower than 10 bar. The catalyst to be used is not essential and may be a solid acid catalyst, such as a zeolite. Said step may be carried out in the gas phase or in the liquid phase, preferably in the gas phase. Before performing the next step in the present process, preferably water is removed. Further, preferably, any unconverted starting material and/or any di- and tri-acylated products are removed before performing the next step.

In a next step of the present process for producing styrene, the methyl phenyl ketone thus obtained is converted into styrene.

Preferably, in the present process for producing styrene, the methyl phenyl ketone is converted into styrene via the following two reactions. In a first reaction, the methyl phenyl ketone is converted into methyl phenyl carbinol by hydrogenation, as follows:

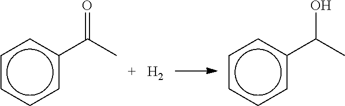

In a second reaction, said methyl phenyl carbinol is converted into styrene by dehydration, as follows:

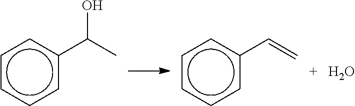

Thus, in the present process, it is preferred that the methyl phenyl ketone is converted into styrene by converting the methyl phenyl ketone into methyl phenyl carbinol and converting the methyl phenyl carbinol into styrene.

Both the conversion of methyl phenyl ketone into methyl phenyl carbinol as such and the conversion of methyl phenyl carbinol into styrene as such are known. For example, in the above-mentioned SM/PO process, ethylbenzene hydroperoxide is reacted with propylene to yield propylene oxide and methyl phenyl carbinol. In a next step, the methyl phenyl carbinol is converted into styrene. In this process, part of the ethylbenzene hydroperoxide rearranges into methyl phenyl ketone as follows:

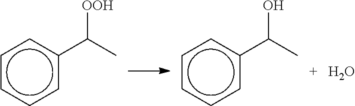

This has the result that the reaction mixture obtained by reacting ethylbenzene hydroperoxide with propylene not only contains propylene oxide and methyl phenyl carbinol, but also methyl phenyl ketone. Said methyl phenyl ketone cannot be dehydrated in the next step wherein styrene is produced from methyl phenyl carbinol. After the latter step, the methyl phenyl ketone has to be separated and then it is hydrogenated resulting in methyl phenyl carbinol which is then subjected to dehydration resulting in styrene as yet.

An advantage of the present styrene production process is that the final step of converting the methyl phenyl ketone into styrene can be integrated with the above-mentioned SM/PO process wherein methyl phenyl ketone is also converted into styrene. Therefore, preferably, the present styrene production process additionally comprises:

converting a mixture comprising ethylbenzene hydroperoxide and propylene into a mixture comprising propylene oxide, methyl phenyl carbinol and methyl phenyl ketone,

separating propylene oxide from the mixture comprising propylene oxide, methyl phenyl carbinol and methyl phenyl ketone resulting in a mixture comprising methyl phenyl carbinol and methyl phenyl ketone,

converting the mixture comprising methyl phenyl carbinol and methyl phenyl ketone into a mixture comprising styrene and methyl phenyl ketone,

separating methyl phenyl ketone from the mixture comprising styrene and methyl phenyl ketone,

converting the separated methyl phenyl ketone into methyl phenyl carbinol and combining said methyl phenyl carbinol with the mixture comprising methyl phenyl carbinol and methyl phenyl ketone resulting from separating propylene oxide from the mixture comprising propylene oxide, methyl phenyl carbinol and methyl phenyl ketone,

wherein the methyl phenyl ketone resulting from the reaction of benzene with acetic acid is combined with the methyl phenyl ketone separated from the mixture comprising styrene and methyl phenyl ketone.

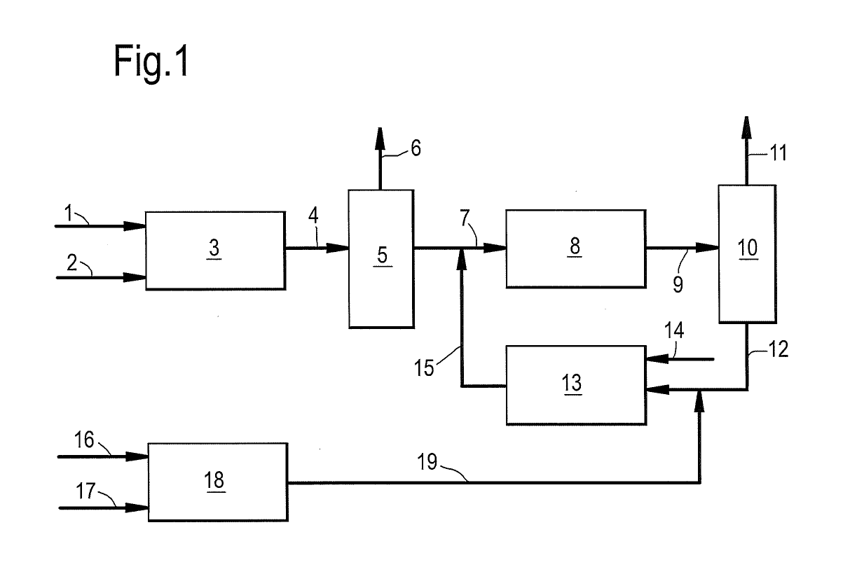

The above embodiment wherein the present styrene production process is integrated with the above-mentioned SM/PO process is exemplified in FIG. 1. In FIG. 1, ethylbenzene hydroperoxide and propylene are sent via lines 1 and 2, respectively, to reaction unit 3 wherein they are converted into a mixture comprising propylene oxide, methyl phenyl carbinol and methyl phenyl ketone. Said mixture comprising propylene oxide, methyl phenyl carbinol and methyl phenyl ketone may also comprise unconverted propylene which may be separated from said mixture and recycled to reaction unit 3 (not shown in FIG. 1). Said mixture comprising propylene oxide, methyl phenyl carbinol and methyl phenyl ketone is sent via line 4 to separation unit 5 wherein propylene oxide is separated via line 6 from the mixture comprising propylene oxide, methyl phenyl carbinol and methyl phenyl ketone resulting in a mixture comprising methyl phenyl carbinol and methyl phenyl ketone. Said mixture comprising methyl phenyl carbinol and methyl phenyl ketone is sent via line 7 to reaction unit 8 wherein it is converted into a mixture comprising styrene and methyl phenyl ketone. Said mixture comprising styrene and methyl phenyl ketone may also comprise water which may be separated from said mixture (not shown in FIG. 1). Said mixture comprising styrene and methyl phenyl ketone is sent via line 9 to separation unit 10 wherein styrene and methyl phenyl ketone are separated from the mixture comprising styrene and methyl phenyl ketone via lines 11 and 12, respectively. The separated methyl phenyl ketone is sent via line 12 to reaction unit 13 wherein it is converted into methyl phenyl carbinol by means of hydrogen provided to reaction unit 13 via line 14. Said methyl phenyl carbinol is sent via line 15 to line 17 where it is combined with the mixture comprising methyl phenyl carbinol and methyl phenyl ketone resulting from separating propylene oxide from the mixture comprising propylene oxide, methyl phenyl carbinol and methyl phenyl ketone in separation unit 5. Further, in FIG. 1, benzene and acetic acid are sent via lines 16 and 17, respectively to reaction unit 18 wherein they are converted into methyl phenyl ketone. Said methyl phenyl ketone is sent via line 19 to line 12 where it is combined with the methyl phenyl ketone separated from the mixture comprising styrene and methyl phenyl ketone in separation unit 10.

In the present invention, the methyl phenyl ketone may be converted into styrene in any known way. More specifically, in an embodiment of the present invention as described above, methyl phenyl ketone may be converted into methyl phenyl carbinol and said methyl phenyl carbinol may then be converted into styrene in any known ways. Likewise, in an embodiment of the present invention as described above, ethylbenzene hydroperoxide and propylene may be converted into propylene oxide, methyl phenyl carbinol and methyl phenyl ketone in any known way. More particularly, the reaction conditions that are known in relation to the above-mentioned SM/PO process for effecting said reactions may equally be applied in the present styrene production process.

The reaction conditions under which said conversions may be carried out, such as catalyst, temperature, pressure, etc., are not essential for obtaining the advantages of the present invention. For illustration purposes only, some reaction conditions are exemplified hereinbelow.

For example, said conversion of methyl phenyl ketone (MPK) into methyl phenyl carbinol may be carried out by treatment with an excess of hydrogen (H₂), wherein the molar ratio of H₂ to MPK is higher than 1:1, at a pressure of from 5 to 100 bar, preferably 10 to 90 bar. The catalyst may be a catalyst containing at least one transition metal, such as copper (Cu), chromium (Cr) and/or zinc (Zn), preferably Cu. Preferably, the catalyst is a supported catalyst. Suitable catalysts are a copper chromite (CuCr₂O₃) catalyst, a catalyst containing copper supported on silica or alumina, and a catalyst comprising CuZn. The temperature may be of from 50 to 200° C., preferably 70 to 150° C.

Further, for example, said conversion of methyl phenyl carbinol into styrene may be carried out in the gas phase at a temperature of from 150 to 450° C., preferably 250 to 350° C. and at a pressure of from 0.1 to 2 bar, preferably 0.5 to 1.5 bar, by using a titania, alumina or zeolite catalyst. Said alumina catalyst may be moderated by an alkali metal. When using a zeolite catalyst, said conversion may alternatively be carried out in the liquid phase, for example at a temperature of from 100 to 200° C. Suitable conditions for effecting said conversion are disclosed by J. K. F. Buijink et al. in Section 3.3 (“Catalytic dehydration”) from Chapter 13 (“Propylene Epoxidation via Shell's SMPO Process: 30 Years of Research and Operation”) from “Mechanisms in Homogeneous and Heterogeneous Epoxidation Catalysis”, edited by T. Oyama, Elsevier, 2008, pages 367-369, the entire disclosure of which is herein incorporated by reference.

Still further, for example, said conversion of ethylbenzene hydroperoxide (EBHP) and propylene into propylene oxide, methyl phenyl carbinol and methyl phenyl ketone may be carried out in the liquid phase at a temperature of from 30 to 200° C., preferably 50 to 150° C. and at a pressure of from 10 to 100 bar, preferably 30 to 70 bar. Propylene may be used in excess. The molar ratio of propylene to EBHP may be of from 2 to 10, typically 3 to 8. Further, preferably, the catalyst is a titanium containing catalyst, which is preferably supported on silica. The latter catalyst may be prepared in a multistep gas-phase process by treatment of a silica carrier with titanium tetrachloride, heating the obtained material, followed by steaming and silylation. Suitable conditions for effecting said conversion are disclosed by J. K. F. Buijink et al. in Section 2 (“Catalytic Epoxidation”) from Chapter 13 (“Propylene Epoxidation via Shell's SMPO Process: 30 Years of Research and Operation”) from “Mechanisms in Homogeneous and Heterogeneous Epoxidation Catalysis”, edited by T. Oyama, Elsevier, 2008, pages 358-362, the entire disclosure of which is herein incorporated by reference.

1. A process for producing styrene, comprising reacting benzene and acetic acid into methyl phenyl ketone and converting the methyl phenyl ketone into styrene.

2. A process according to claim 1, wherein the methyl phenyl ketone is converted into styrene by converting the methyl phenyl ketone into methyl phenyl carbinol and converting the methyl phenyl carbinol into styrene.

3. A process according to claim 1, additionally comprising:
converting a mixture comprising ethylbenzene hydroperoxide and propylene into a mixture comprising propylene oxide, methyl phenyl carbinol and methyl phenyl ketone,
separating propylene oxide from the mixture comprising propylene oxide, methyl phenyl carbinol and methyl phenyl ketone resulting in a mixture comprising methyl phenyl carbinol and methyl phenyl ketone,
converting the mixture comprising methyl phenyl carbinol and methyl phenyl ketone into a mixture comprising styrene and methyl phenyl ketone,
separating methyl phenyl ketone from the mixture comprising styrene and methyl phenyl ketone,
converting the separated methyl phenyl ketone into methyl phenyl carbinol and combining said methyl phenyl carbinol with the mixture comprising methyl phenyl carbinol and methyl phenyl ketone resulting from separating propylene oxide from the mixture comprising propylene oxide, methyl phenyl carbinol and methyl phenyl ketone,
wherein the methyl phenyl ketone resulting from the reaction of benzene with acetic acid is combined with the methyl phenyl ketone separated from the mixture comprising styrene and methyl phenyl ketone.

4. A process according to claim 1, wherein the acetic acid and/or benzene originates from a biomass conversion process.

5. A process according to claim 1, wherein the acetic acid originates from a cellulosic material, such as a lignocellulosic material.
